(19) United States
(12) Patent Application Publication
(10) Pub. No.: US 2005/0267017 A1
(43) Pub. Date: Dec. 1, 2005

(54) Conformationally constrained C-backbone cyclic peptides

(76) Inventors: Chaim Gilon, Jerusalem (IL); Yaniv Barda, Talme Menashe (IL)

Correspondence Address: WINSTON & STRAWN LLP, 1700 K STREET, N.W., WASHINGTON, DC, 20006, US

(21) Appl. No.: 11/085,906

(22) Filed: Mar. 21, 2005

Related U.S. Application Data
(63) Continuation of application No. 10/882,811, filed on Jul. 2, 2004.
Application No. 10/882,811 is a continuation of application No. 10/882,611, filed on Jul. 2, 2004, now abandoned.
Application No. 10/882,611 is a continuation of application No. PCT/IL03/00006, filed on Jan. 2, 2003.
(60) Provisional application No. 60/344,037, filed on Jan. 3, 2002.

(51) Int. Cl. A61K 38/12; C07K 7/64

(52) U.S. Cl. 514/9; 530/317

(57) ABSTRACT

Backbone cyclized peptide analogs that include at least one building unit of a C^{α}(ω-functionalized) amino acid that is constructed to include a spacer and a terminal functional group. The bridging groups are attached via alpha carbons of amino acid derivatives to provide novel non-peptide linkages. Also disclosed are novel C^{α}(ω-functionalized) amino acid building units, and methods of preparing them as well as the cyclized peptide analogs, preferably during solid phase peptide synthesis. The reactive terminal functional groups are protected by specific protecting groups that can be selectively removed to effect the desired cyclization.

### CROSS-REFERENCE TO RELATED APPLICATIONS

This application is a continuation of U.S. application Ser. No. 10/882,611 filed Jul. 2, 2004, which is a continuation of International application PCT/IL03/00006 filed Jan. 2, 2003, and which claims the benefit of U.S. provisional application 60/344,037 filed Jan. 3, 2002, the entire content of each of which is expressly incorporated herein by reference.

### FIELD OF THE INVENTION

The present invention relates to a novel class of conformationally constrained C^{α}-backbone-cyclized peptide analogs, to a process for their preparation from C^{α}-functionalized-alkyl-amino acid building units, to certain novel C^{α}ω-functionalized-alkyl-amino acid building blocks, and to a process for the preparative scale synthesis of the C^{α}ω-functionalized-alkyl-amino acid building units. The present invention further relates to pharmaceutical compositions comprising the novel cyclic peptides and to methods of use thereof.

### BACKGROUND OF THE INVENTION

As a result of major advances in organic chemistry and in molecular biology, many bioactive peptides can now be prepared in quantities sufficient for pharmacological and clinical utilities. Thus in the last few years new methods have been established for the treatment and therapy of illnesses in which peptides have been implicated. However, the use of peptides as drugs is limited by the following factors: a) their low metabolic stability towards proteolysis in the gastrointestinal tract and in serum; b) their poor absorption after oral ingestion, in particular due to their relatively high molecular mass or the lack of specific transport systems or both; c) their rapid excretion through the liver and kidneys; and d) their undesired side effects in non-target organ systems, since peptide receptors can be widely distributed in an organism.

Moreover, with a few exceptions, native peptides of small to medium size (less than 30-50 amino acids) exist unordered in dilute aqueous solution in a multitude of conformations in dynamic equilibrium which may lead to lack of receptor selectivity, metabolic susceptibilities and hamper attempts to determine the biologically active conformation. If a peptide has the biologically active conformation per se, i.e., receptor-bound conformation, then an increased affinity toward the receptor is expected, since the decrease in entropy on binding is less than that on the binding of a flexible peptide. It is therefore important to strive for and develop ordered, uniform and biologically active peptides:

In recent years, intensive efforts have been made to develop peptide analogs or peptide analogs that display more favorable pharmacological properties than their prototype native peptides. The native peptide itself, the pharmacological properties of which have been optimized, generally serves as a lead for the development of these peptide analogs. However, a major problem in the development of such agents is the discovery of the active region of a biologically active peptide. For instance, frequently only a small number of amino acids (usually four to eight) are responsible for the recognition of a peptide ligand by a receptor. Once this biologically active site is determined a lead structure for development of peptide analogs can be optimized, for example by molecular modeling programs.

As used herein, a “peptide analog” or “peptide analog” is a compound that, can mimic (as an “agonist”) or block (as an “antagonist”) a biologically active peptide that constitutes an epitope or binding site or regulatory element as one member of a recognition forming group involved in intermolecular interactions (e.g., receptor-ligand, antibody-antigen, enzyme-substrate, nucleic acid sequence-DNA binding protein, etc.). The mimicking or blocking results in modulation of the intermolecular interaction of the member with the other member of the recognition-forming group and resulting in a change in a biological property. The following factors should be considered to achieve the best possible agonist peptide analogs a) metabolic stability, b) good bioavailability, c) high affinity and selectivity to the other member of the recognition forming group (the receptor or ligand, the antibody or the antigen, the enzyme or the substrate, the DNA etc.), and d) minimal side effects.

From the pharmacological and medical viewpoint it is frequently desirable not only to imitate the effect of the peptide at the level of interacting with the other member of the recognition group (agonism) but also to block the biological activity elicited by the interaction with the other member when required (antagonism). The same pharmacological considerations for designing agonist peptide analogs mentioned above hold for designing peptide antagonists, but, in addition, their development in the absence of lead structures is more difficult. Even today it is not unequivocally clear which factors are decisive for the agonistic effect and which are for the antagonistic effect.

A generally applicable and successful method recently has been the development of conformationally restricted peptide analogs that imitate the receptor-bound conformation of the endogenous peptide ligands as closely as possible (Rizo and Gierasch, Anti. Rev. Biochem. 61:387, 1992). Investigations of these types of analogs show them to have increased resistance toward proteases, that is, an increase in metabolic stability, as well as increased selectivity and thereby fewer side effects (Veber and Friedinger, Trends Neurosci., p. 392, 1985).

Once these peptide analogs compounds with rigid conformations are produced, the most active structures are selected by studying the conformation-activity relationships. Such conformational constraints can involve short range (local) modifications of structure or long range (global) conformational restraints (for review see Giannis and Kolter, Angew. Chem. Int. Ed. Engl. 32:1244, 1993).

Conformationally Constrained Peptides

Bridging between two neighboring amino acids in a peptide leads to a local conformational modification, the flexibility of which is limited in comparison with that of regular dipeptides. Some possibilities for forming such bridges include incorporation of lactams and piperazinones. γ-Lactams and δ-lactams have been designed to some extent as “turn mimetics”; in several cases the incorporation of such structures into peptides leads to biologically active compounds.

Global restrictions in the conformation of a peptide are possible by limiting the flexibility of the peptide strand through cyclization (Hruby et al., Biochem. J., 268:249, 1990). Not only does cyclization of bioactive peptides improve their metabolic stability and receptor selectivity, cyclization also imposes constraints that enhance conformational homogeneity and facilitates conformational analysis.

In addition, conformationally constrained amino acids and amino acid derivatives have ubiquitous use as building units for the construction of peptides and small molecules combinatorial libraries. These libraries contain proteinous pharmacophors, or their mimics, since they are aimed to bring about the discovery of drug leads that will inhibit protein:protein and protein:nucleic acid interactions. A large majority of the peptide and small molecule libraries are cyclic, since cyclization improves the pharmacological properties, such as selectivity and ADME, of the lead.

The common modes of cyclization are the same found in naturally occurring cyclic peptides. These include side chain to side chain cyclization or side chain to end-group cyclization. For this purpose, amino acid side chains that are not involved in receptor recognition are connected together or to the peptide backbone. Another common cyclization is the end-to-end cyclization.

Three representative examples are compounds wherein partial structures of each peptide are made into rings by linking two penicillamine residues with a disulfide bridge (Mosberg et al., P.N.A.S. US, 80:5871, 1983), by formation of an amide bond between a lysine and an aspartate group (Charpentier et al., J. Med. Chem. 3: 1184, 1989), or by connecting two lysine groups with a succinate unit (Rodriguez et al., Int. J. Pept. Protein Res. 35:441, 1990). These structures have been disclosed in the literature in the case of a cyclic enkephalin analog with selectivity for the .delta.-opiate receptor (Mosberg et al., ibid.); or as agonists to the cholecystokinin B receptor, found largely in the brain (Charpentier et al., ibid., Rodriguez et al., ibid.).

One of the capital disadvantages of the classical cyclization methods which include amino end to carboxy end (AE-CE), side chain to side chain (SC-SC), side chain to amino end (SC-AE) and side chain to carboxy end (SC-CE) modes of cyclizations, is the loss of activity by the use of crucial functional groups for cyclization.

Another conceptual approach to the conformational constraint of peptides was introduced by Gilon, et al. [Gilon, C.; Halle, D.; Chorev, M.; Selinger, Z.; Byk, G., (1991) Biopolymers, 31, 745-750] who proposed a new method for conferring long range conformational constraint on peptides, namely N-backbone cyclization (N-BC). N-BC overcomes the insufficiency of the prior art cyclization methods by forming the cyclization from the peptide skeleton, without changing the original functional groups of the peptide. Thus, the four-modes of N-backbone cyclization are: amino end to backbone nitrogen (AE-BN), backbone nitrogen to side chain (BN-SC), backbone nitrogen to backbone nitrogen (BN-BN) and backbone nitrogen to carboxy end (BN-CE). The theoretical advantages of this strategy include the ability to effect cyclization via the carbons or nitrogens of the peptide backbone without interfering with side chains that may be crucial for interaction with the specific receptor of a given peptide.

While the concept was envisaged as being applicable to any linear peptide of interest, the limiting factor in the method of Gilon et al. (EP 564,739; and J. Org. Chem., 57:5687, 1992) was the availability of suitable building units that must be used to replace the amino acids that are to be linked via bridging groups.

Subsequently, a series of building units for N^{α} backbone cyclization (N-BU) suitable for solid phase synthesis were prepared [Muller, D., Zeltser, I., Bitan, G. and Gilon, C., (1996) J. Org. Chem., 62, 411-416; Bitan et al., (1997) J. Pept. Res., 49, 421-426; Gellerman et al., (2001) J. Peptide Res., 57, 277-291; Gazal et al., (2001) J Pept. Res., 58, 527-539; Gazal et al., (2002) J Med. Chem., 45, 1665-1671] (FIG. 1A).

U.S. Pat. Nos. 5,723,575; 5,811,392; 5,874,529; 5,883,293; 6,117,974 and 6,265,375, commonly assigned to the assignees of the present invention disclose, a series of N-backbone cyclized peptide analogs formed by means of bridging groups attached via the alpha nitrogens of amino acid derivatives to provide novel non-peptidic linkages, and libraries of these backbone-cyclized peptide analogs. Novel building units disclosed are N^{α}(ω-functionalized) amino acids constructed to include a spacer and a terminal functional group. One or more of these N^{α}(ω-functionalized) amino acids are incorporated into a peptide sequence, preferably during solid phase peptide synthesis. The reactive terminal functional groups are protected by specific protecting groups that can be selectively removed to effect either backbone-to-backbone or backbone-to-side chain cyclizations.

In order to accelerate the processes of discovering a lead N-BC compound that have the desired biological activity, a method called cycloscan [Gilon, C.; Kessler, H., (2002) Curr. Opin. in Chem. and Biol., in press] was introduced. Cycloscan comprises the preparation of backbone cyclic peptide libraries and their screening with the appropriate biological assay. All the members of the cycloscan library have the same primary sequence and they differ from each other only in one variable such as the mode of cyclization, the location of the ring, the ring size and the ring chemistry.

Nowhere in the background are there any synthetic examples of C^{α} backbone cyclized peptide analogs, other than hypothetical structures without any operative methods of making them.

### SUMMARY OF THE INVENTION

The present invention provides novel backbone cyclized peptide analogs comprising at least one building unit, which is a C^{α}(ω-functionalized) amino acid constructed to include a spacer and a terminal functional group. The cyclized peptide analogs are formed by means of bridging groups attached via the alpha-carbons of amino acid derivatives to provide novel non-peptidic linkages. The present invention further provides certain novel C^{α}(ω-functionalized) amino acid building units, methods of preparing the C^{α}(ω-functionalized) amino acid building units, and methods of preparing the novel backbone cyclized peptide analogs by incorporating one or more of these C^{α}(ω-functionalized) amino acid building units into a peptide sequence, preferably during solid phase peptide synthesis. The reactive terminal functional groups are protected by specific protecting groups that can be selectively removed to effect either backbone-to-backbone or backbone-to-side chain cyclizations.

Thus, it is an object of the present invention to provide a cyclized peptide analog comprising a sequence of amino acids that incorporates at least one building unit which is a modified amino acid having an alpha-carbon atom of the peptide backbone attached through an optional spacer to a functional group selected from amine, thio, oxy, and carboxy. The building unit is joined to another amino acid within the peptide sequence to form a bridging group comprising a disulfide, amide, thioether, thioester, imine, ether, ester or an alkene.

The building units may be present at one or both end of the sequence of amino acids or alternatively may be positioned in non-terminal positions of the sequence.

In one embodiment, the cyclized peptide analog comprises two building units joined together to form a cyclic structure. In another embodiment, the cyclized peptide analog comprises one building unit.

The building units are joined to another amino acid within the sequence through five optional modes of cyclization: a) building unit to an amino acid located at the carboxy end of the peptide sequence (C-backbone to carboxy end); b) building unit to an amino acid located at the amino end of the peptide sequence (C-backbone to amino end); c) building unit to an amino acid through the side chain of the amino acid (C-backbone to side chain); d) building unit to another building unit (C-backbone to C-backbone); and/or e) building unit to an amino acid through the backbone nitrogen of the amino acid (C-backbone to N-backbone).

Thus, according to one aspect of the present invention, cyclized peptide analogs are provided that have the general Formula (I): 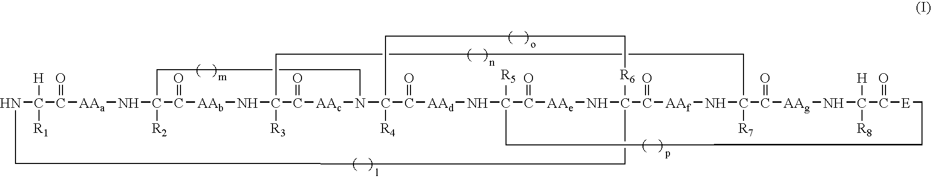
wherein
a, b, c, d, e, f and g are independently of each other an integer from 1 to 8 or zero;
l, m, n, o and p are independently of each other zero or 1, wherein at least one of l, m, n, o or p is 1;
each AA designates an amino acid residue wherein the amino acid residues may be the same or different;
E designates an oxygen, an amino, a carboxyl protecting group, wherein E is optionally bound to a solid support, or CO-E can be reduced to CH₂O;
R₁-R₈ are independently of each other hydrogen or an amino acid side-chain optionally bound with a protecting group; and
   the lines designate a bridging group of the Formula:
      (i) —X-M-Y—W-Z- or (ii) —X-M-Z-
wherein
   M and W are independently of each other a disulfide, amide, thioether, thioester, imine, ether, ester or an alkene; and
   X, Y and Z independently of each other an unsubstituted or substituted alkylene, alkenylene, alkynylene, arylene, cycloalkylene, alkylarylene, heterocycloalkylene or heteroarylene.
      In certain preferred embodiments, the CO-E group of Formula (I) is reduced to a CH₂O group.

In accordance with another preferred embodiment of the present invention, the cyclized peptide analog is represented by the structure of Formula (II): 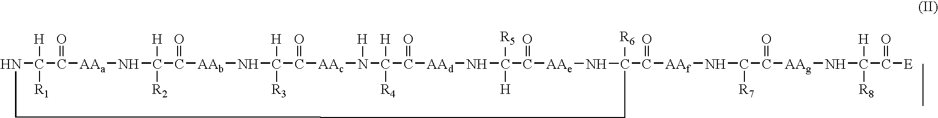
wherein the substituents are as defined above.

In accordance with another preferred embodiment of the present invention, the cyclized peptide analog is represented by the structure of Formula (III): 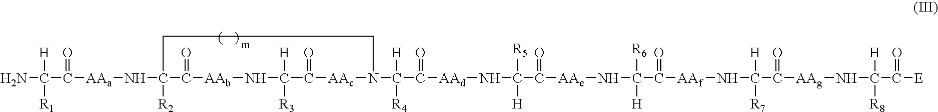
wherein the substituents are as defined above.

In accordance with another preferred embodiment of the present invention, the cyclized peptide analogs peptide analog is represented by the structure of Formula (IV): 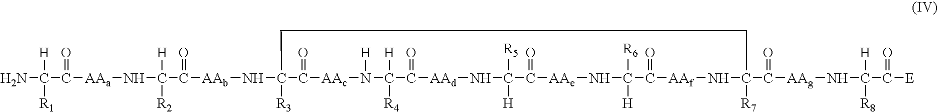
wherein the substituents are as defined above.

In accordance with another preferred embodiment of the present invention, the cyclized peptide analog is represented by the structure of Formula (V): 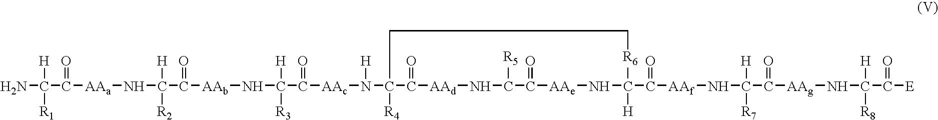
wherein the substituents are as defined above.

In accordance with another preferred embodiment of the present invention, the cyclized peptide analog is represented by the structure of Formula (VI): 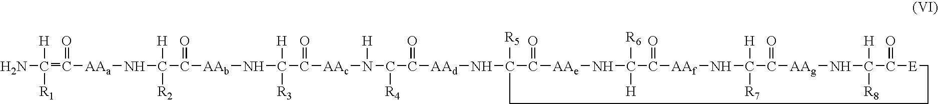
wherein the substituents are as defined above.

A preferred embodiment of the present invention involves the cyclized peptide analog of Formulae I-VI wherein the line designates a bridging group of the Formula: —(CH₂)ₓ-M-(CH₂)_{y}—W—(CH₂)_{z}—, wherein M and W are independently of each other a disulfide, amide, thioether, thioester, imine, ether, ester or an alkene; x and z each independently designates an integer from 1 to 10, and y is zero or an integer of from 1 to 8, with the proviso that if y is zero, W is absent.

Further preferred are backbone cyclized peptide analogs of the any of formulas I-VI wherein R₁-R₈ are independently of each other CH₃—, (CH₃)₂—CH—, (CH₃)₂—CHCH₂—, CH₃CH₂CH(CH₃)—, CH₃S(CH₂)₂—, HOCH₂—, CH₃CH(OH)—, HSCH₂—, NH₂C(═O)CH—, NH₂C(═O), (CH₂)₂—, —NH₂(CH₂)₃—, HOC(═O)CH₂—, HOC(═O)(CH₂)₂—, NH₂(CH₂)₄—, C(NH)₂NH(CH₂)₃—, HO-phenyl-CH₂—, benzyl, methylindole, or methylimidazole.

The backbone peptides of the present invention are prepared from ω-functionalized amino acid derivative of the general Formula X: 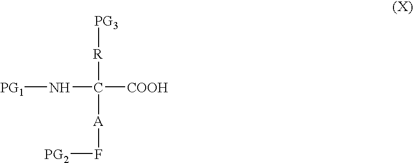
wherein
   A is a spacer group selected from unsubstituted or substituted alkylene, alkenylene, alkynylene, arylene, cycloalkylene, alkylarylene, heterocycloalkylene or heteroarylene;
   F is a functional group selected from amine, thio, oxy, or carboxy;
   PG₁, PG₂ and PG₃ are independently of each other hydrogen or a protecting group selected from alkyloxy, substituted alkyloxy, or aryloxy carbonyls; and
   R is a side chain of an amino acid.

Preferred building units are the (i)-functionalized amino acid derivatives wherein A is alkylene. Further preferred are ω-functionalized amino acid derivatives wherein R is protected with a specific protecting group PG₃.

Another aspect of the present invention is directed to novel ω-functionalized amino acid derivatives of formula X, which include amino acids having natural or unnatural side chains, such as the following, non-limiting examples: CH₃—, (CH₃)₂—CH—, (CH₃)₂—CHCH₂—, CH₃CH₂CH(CH₃)—, CH₃S(CH₂)₂—, HOCH₂—, CH₃CH(OH)—, HSCH₂—, NH₂C(═O)CH₂—, NH₂C(═O), (CH₂)₂—, NH₂(CH₂)₃—, HOC(═O)CH₂—, HOC(═O)(CH₂)₂—, NH(CH₂)₄—, C(NH₂)₂NH(CH₂)₃—, HO-phenyl-CH₂—, benzyl, methylindole, or methylmidazole.

Another aspect of the present invention, is directed to a method for pre-paring, the ω-functionalized amino acid derivatives of formula X by reacting a carboxylic acid derivative of formula VII with a reagent containing a nucleophilic R group, to produce compound VIII; converting compound VIII to amino acid derivative IX; and optionally protecting the amino group of compound IX; thereby preparing the ω-functionalized amino acid derivative X.

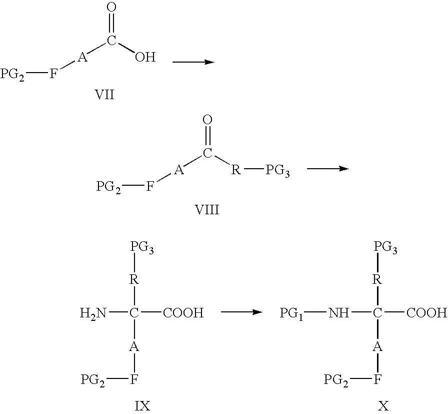

The step of converting carboxylic acid VII to compound VIII may be carried out by any mode known to a person skilled in the art. In a preferred embodiment, this step comprises initially converting the carboxylic acid into a reactive derivative thereof; and reacting, the reactive carboxylic acid derivative with a compound containing a nucleophillic R group. In a particularly preferred embodiment, this step is carried out under conditions of the Weinreb reaction.

In another preferred embodiment, the step of converting compound VIII to compound IX is carried out under conditions of the Strecker synthesis.

In one embodiment, PG₁ is an amino protecting group. In another embodiment, PG₂ is a functional group (F) protecting group. In another embodiment, PG₃ is a side chain protecting group. The protecting groups are selected from Ada, Aloc, Allyl, Boc, Bzl, Fmoc, OBzl, OEt, OMe, Tos, Trt and benzyloxycarbonyl.

In one embodiment, the compound containing the nucleophillic R group is represented by the structure RM(L)ₓ wherein M is a metal, L is a leaving group and X is zero or 1.

A further aspect of this invention is to provide methods for the preparation of novel backbone cyclic peptides, comprising the steps of incorporating at least one C^{α}-ω-functionalized derivatives of amino acids into a peptide sequence and subsequently selectively cyclizing the functional group with one of the side chains of the amino acids in the peptide sequence, or with another ω-functionalized amino acid derivative. Thus, in one aspect, the present invention provides a method for the preparation of a cyclized peptide analog of the general Formula (I): 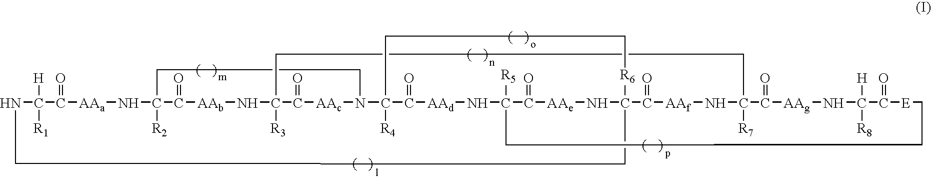
wherein
a, b, c, d, e, f and g are independently of each other an integer from 1 to 8 or zero;
l, m, n, o and p are independently of each other zero or 1, wherein at least one of l, m, n, o or p is 1;
each AA designates an amino acid residue wherein the amino acid residues may be the same or different;
E designates an oxygen, an amino, a carboxyl protecting group, wherein E is optionally bound to a solid support, or CO-E can be reduced to CH₂O;
R₁-R₈ are independently of each other hydrogen or an amino acid side-chain optionally bound with a protecting group; and
the lines designate a bridging group of the Formula:
   (i) —X-M-Y—W-Z- or (ii) —X-M-Z-
wherein
   M and W are independently of each other a disulfide, amide, thioether, thioester, imine, ether, ester or an alkene; and
   X, Y and Z independently of each other an unsubstituted or substituted alkylene, alkenylene, alkynylene, arylene, cycloalkylene, alkylarylene, heterocycloalkylene or heteroarylene.
by incorporating at least one C^{α}-ω-functionalized derivatives of amino acid of Formula (X) into a peptide sequence and subsequently selectively cyclizing the functional group with one of the side chains of the amino acids in the peptide, with the carboxy end of the peptide, with the amino end of the peptide, with another amino acid through the backbone nitrogen of the amino acid, or with another C^{α}-ω-functionalized amino acid derivative 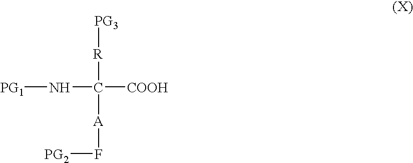
wherein
A is a spacer group selected from unsubstituted or substituted alkylene, alkenylene, alkynylene, arylene, cycloalkylene, alkylarylene, heterocycloalkylene or heteroarylene;
F is a functional group selected from amine, thio, oxy, or carboxy;
PG₁, PG₂ and PG₃ are independently of each other hydrogen or a protecting group selected from alkyloxy, substituted alkyloxy, or aryloxy carbonyls; and
R is a side chain of an amino acid.

Backbone cyclized analogs of the present invention may be used as pharmaceutical compositions and in methods for the treatment of disorders including in the treatment of cardiovascular, cerebrovascular, dermatological, endocrine, gastrointestinal, gynecological, hematological, hepatic, hormonal, immunological, metabolic, muscular, neural, neurological, ophthalmologic, pulmonary, renal, skeletal, and urological disorders and diseases. Pharmaceutical preparations containing C-backbone cyclized peptide analogs are further useful for the treatment of wounds, pain, allergies, and infectious diseases, and for the regulation of immune functions.

Therefore, further objects of the present invention are directed to pharmaceutical compositions comprising pharmacologically active backbone cyclized peptide analogs (which may be agonists or antagonists) prepared according to the methods disclosed herein and a pharmaceutically acceptable carrier or diluent for the treatment, prevention or diagnosis of disease and disorders in humans and animals, and provides methods for the treatment of cancer, metabolic disorders, autoimmune diseases, inflammation, septic shock, neurological diseases and disorders, infectious diseases, cardiopulmonary diseases, asthma or endocrine disorders and gastrointestinal disorders therewith.

### BRIEF DESCRIPTION OF THE DRAWINGS

The present invention will be understood and appreciated more fully from the following detailed description taken in conjunction with the appended drawings in which:

FIG. 1: Building units for backbone cyclization. A: building units for N-backbone cyclization; B: building units for C-backbone cyclization. R=amino acid side chain, F=functional group; n=1-6; PG₁=N^{α} protecting group; PG₂=F protecting group; PG₃=side chain protecting group.

FIG. 2: Modes of C-backbone cyclization: BC-BN=backbone C to backbone N; BC-SC=backbone C to side chain; BC-BC=backbone C to backbone C; BC-CE=backbone C to carboxy end; BC-AE=backbone C to amino end.

FIG. 3: Retro synthetic scheme of protected C^{α}(ω-functional alkylene) amino acids.

FIG. 4: Synthesis of C^{α}(amino ethyl) amino acids.

FIG. 5: Synthesis of Fmoc-C^{α}(Boc-amino ethyl) amino acids.

FIG. 6: Synthesis of Fmoc-C^{α}(Aloc-amino ethyl) phenyl alanine.

### DETAILED DESCRIPTION OF THE INVENTION

The present invention provides novel backbone cyclized peptide analogs comprising at least one building unit which is a C^{α}(ω-functionalized) amino acid constructed to include a spacer and a terminal functional group. The cyclized peptide analogs are formed by means of bridging groups attached via the alpha-carbons of amino acid derivatives to provide novel non-peptidic linkages. The present invention further provides novel C^{α}(ω-functionalized) amino acid building units, methods of preparing the C^{α}(ω-functionalized) amino acid building units, and methods of preparing the novel backbone cyclized peptide analogs by incorporating one or more of these C^{α}(ω-functionalized) amino acid building units into a peptide sequence, preferably during solid phase peptide synthesis. The reactive terminal functional groups are protected by specific protecting groups that can be selectively removed to effect either backbone-to-backbone or backbone-to-side chain cyclizations. 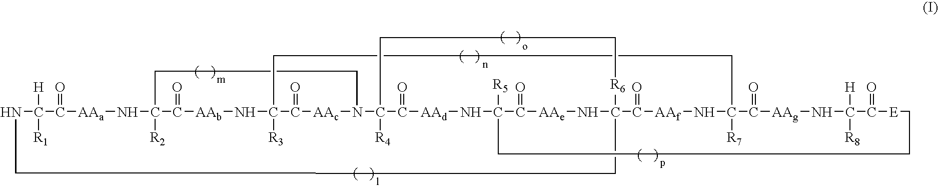
wherein
a, b, c, d, e, f and g are independently of each other an integer from 1 to 8 or zero;
l, m, n, o and p are independently of each other zero or 1, wherein at least one of l, m, n, o or p is 1;
each AA designates an amino acid residue wherein the amino acid residues may be the same or different;
E designates an oxygen, an amino, a carboxyl protecting group, wherein E is optionally bound to a solid support, or CO-E can be reduced to CH₂O;
R₁-R₈ are independently of each other hydrogen or an amino acid side-chain optionally bound with a protecting group; and
the lines designate a bridging group of the Formula:
   (i) —X-M-Y—W-Z- or (ii) —X-M-Z-
wherein
   M and W are independently of each other a disulfide, amide, thioether, thioester, imine, ether, ester or an alkene; and
   X, Y and Z independently of each other an unsubstituted or substituted alkylene, alkenylene, alkynylene, ar-lene, cycloalkylene, alkylarylene, heterocycloalkylene or heteroarylene.

In certain preferred embodiments, the CO-E group of Formula (I) is reduced to a CH₂O group.

In accordance with another preferred embodiment of the present invention, the cyclized peptide analog is represented by the structure of Formula (II): 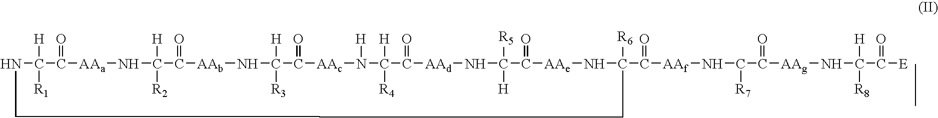
wherein the substituents are as defined above.

In accordance with another preferred embodiment of the present invention, the cyclized peptide analog is represented by the structure of Formula (III): 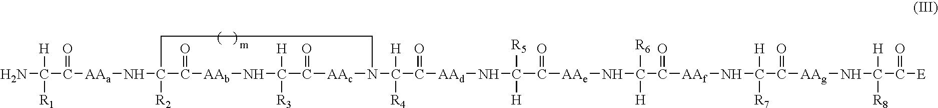
wherein the substituents are as defined above.

In accordance with another preferred embodiment of the present invention, the cyclized peptide analogs peptide analog is represented by the structure of Formula (IV): 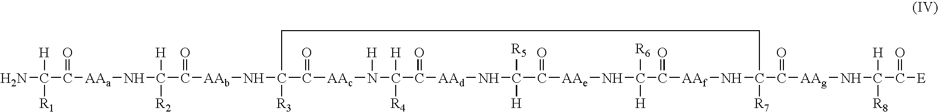
wherein the substituents are as defined above.

In accordance with another preferred embodiment of the present invention, the cyclized peptide analog is represented by the structure of Formula (V): 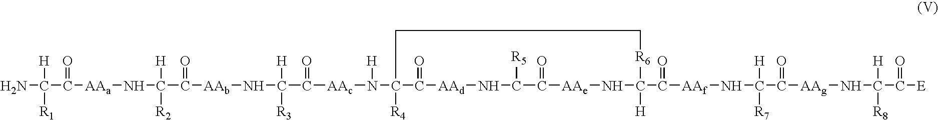
wherein the substituents are as defined above.

In accordance with another preferred embodiment of the present invention, the cyclized peptide analog is represented by the structure of Formula (VI): 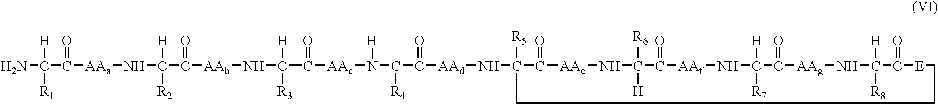
wherein the substituents are as defined above.

A preferred embodiment of the present invention involves the cyclized peptide analog of Formulae I-VI wherein the line designates a bridging group of the Formula: —(CH₂)ₓ-M-(CH₂)_{y}—W—(CH₂)_{z}— wherein M and W are independently of each other a disulfide, amide, thioether, thioester, imine, ether, ester or an alkene; x and z each independently designates an integer from 1 to 10, and y is zero or an integer of from 1 to 8, with the proviso that if y is zero, W is absent.

Further preferred are backbone cyclized peptide analogs of the any of formulas I-VI wherein R₁-R₈ are independently of each other is CH₃—, (CH₃)₂—CH—, (CH₃)₂—CHCH₂—, CH₃CH₂CH(CH₃)—, CH₃S(CH₂)₂—, HOCH₂—, CH₃CH(OH)—, HSCH₂—, NH₁₂C(═O)CH₂—, NH₂C(═O), (CH₂)₂—, NH₂(CH₂)₃—, HOC(═O)CH₂—, HOC(═O)(CH₂)₂—, NH₂(CH₂)₄—, C(NH₂)₂NH(CH₂)₃—, HO-phenyl-CH₂—, benzyl, methylindole, or methylimidazole.

The backbone peptides of the present invention are prepared from ω-functionalized amino acid derivative of the general Formula X: 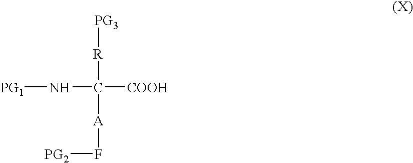
wherein
   A is a spacer group selected from unsubstituted or substituted alkylene, alkenylene, alkynylene, arylene, cycloalkylene, alkylarylene, heterocycloalkylene or heteroarylene;
   F is a functional group selected from amine, thio, oxy, or carboxy;
   PG₁, PG₂ and PG₃ are independently of each other hydrogen or a protecting group selected from alkyloxy, substituted alkyloxy, or aryloxy carbonyls; and
   R is a side chain of an amino acid.

Another aspect of the present invention is directed to novel ω-functionalized amino acid derivatives of formula X, which include amino acids having natural or unnatural side chains, such as the following non-limiting examples: CH₃—, (CH₃)₂—CH—, (CH₃)₂—CHCH₂—, CH₃CH₂CH(CH₃)—, CH₃S(CH₂)₂—, HOCH₂—, CH₃CH(OH)—, HSCH₂—, NH₂C(═O)CH₂—, NH₂C(═O), (CH₂)₂—, NH₂(CH₂)₃—, HOC(═O)CH₂—, HOC(═O)(CH₇)—, NH₂(CH₂)₄—, C(NH₂)₂NH(CH₂)₃—, HO-phenyl-CH₂—, benzyl, methylindole, or methylimidazole.

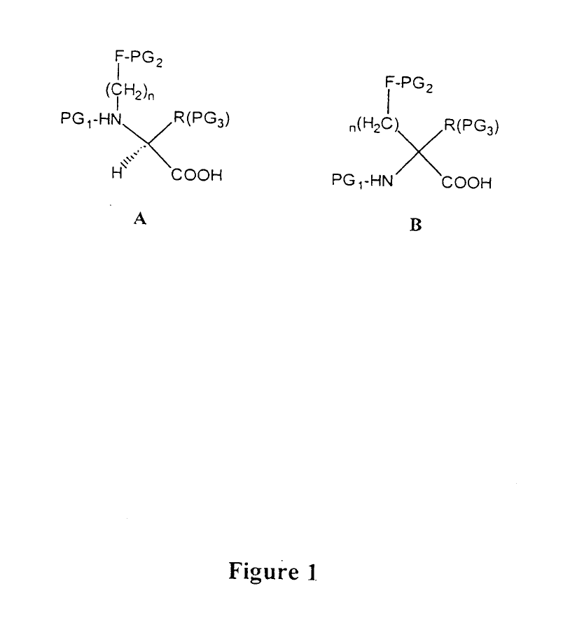

Preferred building units are the ω-functionalized amino acid derivatives wherein A is alkylene. Further preferred are ω-functionalized amino acid derivatives wherein R is protected with a specific protecting group PG₃. A preferred building unit is exemplified in FIG. 1B. The term “substituted” means that hydrogen atoms may be independently replaced by a substituted or unsubstituted alkyl group.

Encompassed within the scope of the present invention is a novel general synthesis of protected C^{α}-ω-functionalized alkylated amino acids (called herein C-BU's) having the general structure shown in FIG. 1B. As contemplated herein, a preparative synthetic method complies with the following requirements:
1. The procedure has to be general, so that it will allow facile synthesis of C-BU's having all the possible lengths of the alkyl chain (various n in FIG. 1B).
2. These new amino acid derivatives should be suitable for SPS (solid phase synthesis). Hence, the various protecting groups PG₁, PG₂ and PG₃ should be orthogonal.
3. The synthetic method should be adapted for large scale synthesis (a few grams in each batch).
4. The synthesis preferably utilizes cheap starting materials and reagents.

As demonstrated herein, Applicants have discovered a general novel synthesis of C-BU's that comply with the guidelines above. The C-BU's described herein derived from amino acids containing non-functional side chain such as alanine, phenylalanine, valine and the non-proteinogenic amino acids: 2-aminobutyric acid and norleucine. (R=Bzl; —CH₃; —CH(CH₃)₂; —(CH₂)₂—CH₃; —(CH₂)₃—CH₃ etc in FIG. 1B) and amine as the functional group for cyclization (F=—NH—).

Thus, in one aspect, the present invention provides a method for preparing the C-BU's of formula X by reacting a carboxylic acid derivative of formula VII with a reagent containing a nucleophillic R group, to produce compound VIII; converting compound VIII to amino acid derivative IX; and optionally protecting the amino group of compound IX; hereby preparing the ω-functionalized amino acid derivative X.

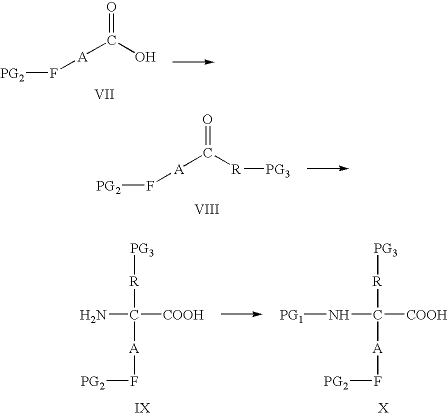

The step of converting carboxylic acid VII to compound VIII may be carried out by any mode known to a person skilled in the art. In a preferred embodiment, this step comprises initially converting the carboxylic acid into a reactive derivative thereof; and reacting the reactive carboxylic acid derivative with a compound containing a nucleophillic R group. In a particularly preferred embodiment, this step is carried out under conditions of the Weinreb reaction. In another preferred embodiment, the step of converting compound VIII to compound IX is carried out under conditions of the Strecker synthesis.

In one embodiment, the compound containing the nucleophillic R group is represented by the structure RM(L), wherein M is a metal, such as lithium, potassium sodium, magnesium and the like, L is a leaving group such as halogen, tosyl and the like, and x is zero or 1. It is appreciated by a person skilled in the art that when M is a monovalent metal, x is 0, and when M is a divalent metal, x is 1.

A further aspect of this invention is to provide methods for the preparation of novel backbone cyclic peptides, comprising the steps of incorporating at least one C^{α}-ω-functionalized derivatives of amino acids into a peptide sequence and subsequently selectively cyclizing the functional group with one of the side chains of the amino acids in the peptide sequence, or with another ω-functionalized amino acid derivative. Thus, in one aspect, the present invention provides a method for the preparation of a backbone cyclized peptide analogs of the general Formula (I): 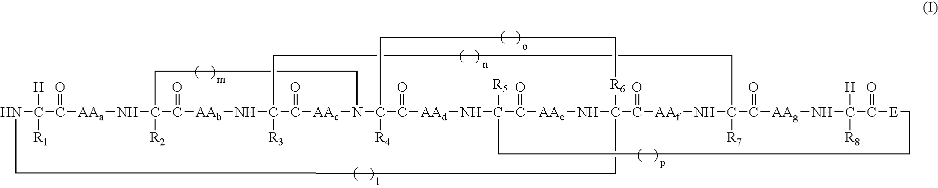
wherein
a, b, c, d, e, f and g are independently of each other an integer from 1 to 8 or zero;
l, m, n, o and p are independently of each other zero or 1, wherein at least one of l, m, n, o or p is 1;
each AA designates an amino acid residue wherein the amino acid residues may be the same or different;
B designates an oxygen, an amino, a carboxyl protecting group, wherein E is optionally bound to a solid support, or CO-E can be reduced to CH₂O;
R₁-R₈ are independently of each other hydrogen or an amino acid side-chain optionally bound with a protecting group; and
the lines designate a bridging group of the Formula:
   (i) —X-M-Y—W-Z- or (ii) —X-M-Z-
wherein
   M and W are independently of each other a disulfide, amide, thioether, thioester, imine, ether, ester or an alkene; and
   X, Y and Z independently of each other an unsubstituted or substituted alkylene, alkenylene, alkynylene, arylene, cycloalkylene, alkylarylene, heterocycloalkylene or heteroarylene,
by incorporating at least one C^{α}-ω-functionalized derivatives of amino acid of Formula (X) into a peptide sequence and subsequently selectively cyclizing the functional group with one of the side chains of the amino acids in the peptide, with the carboxy end of the peptide, with the amino end of the peptide, with another amino acid through the backbone nitrogen of the amino acid, or with another C^{α}-ω-functionalized amino acid derivative. 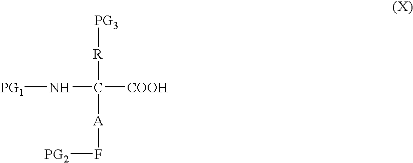
wherein
   A is a spacer group selected from unsubstituted or substituted alkylene, alkenylene, alkynylene, arylene, cycloalkylene, alkylarylene, heterocycloalkylene or heteroarylene;
   F is a functional group selected from amine, thio, oxy, or carboxy;
   PG₁, PG₂ and PG₃ are independently of each other hydrogen or a protecting group selected from alkyloxy, substituted alkyloxy, or aryloxy carbonyls; and
   R is a side chain of an amino acid.
      Definitions:

As used herein a “peptide analog” or “peptide analog”, used herein interchangeably, refers to a no-naturally occurring compound that has structural and chemical similarity to a naturally occurring peptide, which is a member of a recognition forming group (receptor-ligand, enzyme-substrate, antibody-antigen, DINA-DNA binding protein). The analog can mimic the naturally occurring peptide in its interaction with the other member of the recognition group, or alternatively block the naturally occurring peptide, and by those modes modulate the interaction of the native peptide with the other member of the recognition group, leading to a change in the physiological property brought about by the native formation of the recognition group. The modulation may mimic the native interaction and in that case the peptide analog is an “agonist” (this term not only referring to the ligand but to any of the above members of the recognition forming groups) or may interfere with the native interaction and in that case the peptide analog is an “antagonist”.

As used herein, the phrase “an amino acid side chain” refers to the distinguishing substituent attached to the α-carbon of an amino acid; such distinguishing groups are well known to those skilled in the art. For instance, for the amino acid glycine, the R group is H; for the amino acid alanine, R is CH₃, and so on. Other typical side chains of amino acids include the groups: (CH₃)₂CH—, (CH₃)₂CHCH₂—, CH₃CH₂CH(CH₃)—, CH₃S(CH₂)₂—, HOCH₂—, CH₃CH(OH)—, HSCH₂—, NH₂C(═O)CH₂—, NH₂C(═O)(CH₂)₂—, NH₂(CH₂)₃—, HOC(═O)CH₂—, HOC(═O)(CH₂)₂—, NH₂(CH₂)₄—, C(NH₂)₂ NH(CH₂)₃—, HO-phenyl-CH₂—, benzyl, methylindole, and methylimidazole.

As used herein, and in the claims, the letters “(AA),” and the term “amino acid” are intended to include common natural or synthetic amino acids, and common derivatives thereof, known to those skilled in the art, including but not limited to the following. Typical amino-acid symbols denote the L configuration unless otherwise indicated by D appearing before the symbol.

| Abbreviated Designation | Amino Acids |
|---|---|
| Abu | α-Amino butyric acid |
| Ala | L-Alanine |
| Arg | L-Arginine |
| Asn | L-Asparagine |
| Asp | L-Aspartic acid |
| βAsp(Ind) | β-Indolinyl aspartic acid |
| Cys | L-Cysteine |
| Glu | L-Glutamic acid |
| Gln | L-Glutamine |
| Gly | Glycine |
| His | L-Histidine |
| Hyp | trans-4-L-Hydroxy Proline |
| Ile | L-Isoleucine |
| Leu | L-Leucine |
| Lys | L-Lysine |
| Met | L-Methionine |
| Nal | β-Naphthyl alanine |
| Orn | Ornithine |
| Phe | L-Phenylalanine |
| Pro | L-Proline |
| Ser | L-Serine |
| Thr | L-Threonine |
| Trp | L-Tryptophan |
| Tyr | L-Tyrosine |
| Val | L-Valine |

Typical protecting groups (which can be used as PG₁, PG₂ and PG₃ defined hereinabove), coupling agents, reagents and solvents such as but not limited to those listed below have the following abbreviations as used herein. One skill in the art would understand that the compounds listed within each group may be used interchangeably; for instance, a compound listed under “reagents and solvents” may be used as a protecting group, and so on. Further, one skill in the art would know other possible protecting groups, coupling agents and reagents/solvents; these are intended to within the scope of this invention.

Abbreviated Designation

| | |
|---|---|
| | Protecting Groups |
| Ada | Adamantane acetyl |
| Aloc | Allyloxycarbonyl |
| Allyl | Allyl ester |
| Boc | tert-butyloxycarbonyl |
| Bzl | Benzyl |
| Fmoc | Fluorenylmethyloxycarbonyl |
| OBzl | Benzyl ester |
| OEt | Ethyl ester |
| OMe | Methyl ester |
| Tos (Tosyl) | p-Toluenesulfonyl |
| Trt | Triphenylmethyl |
| Z | Benzyloxycarbonyl |
| | Coupling Agents |
| BOP | Benzotriazol-1-yloxytris-(dimethyl |
| | amino)phosphonium hexafluorophosphate |
| DIC | Diisopropylcarbodiimide |
| HBTU | 2-(1HBenzotriazol-1-yl)-1,1,3,3- |
| | tetramethyluronium hexafluorophosphate |
| PyBrOP | Bromotripyrrolidinophosphonium |
| | hexafluorophosphate |
| PyBOP | Benzotriazol-1-yl-oxy-tris-pyrrolidino- |
| | phosphonium hexafluorophosphate |
| TBTU | O-(1,2-dihydro-2-oxo-1-pyridyl)- |
| | N,N,N′,N′-tetramethyluronium |
| | tetrafluoroborate |

The compounds herein described may have asymmetric centers. All chiral, diastereomeric, and racemic forms are included in the present invention. Many geometric isomers of olefins and the like can also be present in the compounds described herein, and all such stable isomers are contemplated in the present invention.

By “stable compound” or “stable structure” is meant herein a compound that is sufficiently robust to survive isolation to a useful degree of purity from a reaction mixture, and Formulation into an efficacious therapeutic agent.

As used herein, “alkyl” is intended to include both branched and straight-chain saturated aliphatic hydrocarbon groups having the C1 to C10 carbon atoms; “alkenyl” is intended to include hydrocarbon chains of either a straight or branched configuration and one or more unsaturated carbon-carbon bonds which may occur in any stable point along the chain, such as ethenyl, propenyl, and the like; and “alkynyl” is intended to include hydrocarbon chains of either a straight or branched configuration and one or more triple carbon-carbon bonds which may occur in any stable point along the chain, such as ethynyl, propynyl, and the like.

As used herein, “aryl” is intended to mean any stable 5- to 7-membered monocyclic or bicyclic or 7- to 14-membered bicyclic or tricyclic carbon ring, any of which may be saturated, partially unsaturated or aromatic, for example, phenyl, naphthyl, indanyl, or tetrahydronaphthyl tetralin, etc.

As used herein, “alkyl halide” is intended to include both branched and straight-chain saturated aliphatic hydrocarbon groups having the C1 to C10 carbon atoms, wherein 1 to 3 hydrogen atoms have been replaced by a halogen atom such as Cl, F, Br, and I.

As used herein, the term “heterocyclic” is intended to mean any stable 5- to 7-membered monocyclic or bicyclic or 7- to 10-membered bicyclic heterocyclic ring, which is either saturated or unsaturated, and which consists of carbon atoms and from 1 to 3 heteroatoms selected from the group consisting of N, O and S and wherein the nitrogen and sulfur atoms may optionally be oxidized, and the nitrogen atom optionally be quaternized, and including any bicyclic group in which any of the above-defined heterocyclic rings is fused to a benzene ring. The heterocyclic ring may be attached to its pendant group at any heteroatom or carbon atom which results in a stable structure. The heterocyclic rings described herein may be substituted on carbon or on a nitrogen atom if the resulting compound is stable. Examples of such heterocycles include, but are not limited to pyridyl, pyrimidinyl, furanyl, thienyl, pyrrolyl, pyrazolyl, imidazolyl, tetrazolyl, benzofuranyl, benzothiophenyl, indolyl, indolenyl, quinolinyl, piperidonyl, pyrrolidinyl, pyrrolinyl, tetrahydrofuranyl, tetrahydroquinolinyl, tetrahydroisoquinolinyl, decahydroquinolinyl, or octahydroisoquinolinyl and the like.

The term, “substituted” as used herein, means that any one or more hydrogen atoms on the designated atom is replaced with a selection from the indicated group, provided that the designated atom's normal valency is not exceeded, and that the substitution results in a stable compound.

When any variable (for example R, x, z, etc.) occurs more than one time in any constituent or in Formulae (I to XX) or any other Formula herein, its definition on each occurrence is independent of its definition at every other occurrence. Also, combinations of substituents and/or variables are permissible only if such combinations result in stable compounds.

Synthetic Approach

According to the present invention peptide analogs are cyclized via bridging groups attached to the alpha carbon atoms of amino acids that permit novel non-peptidic linkages. In general, the procedures utilized to construct such peptide analogs from their building units rely on the known principles of peptide synthesis; most conveniently, the procedures can be performed according to the known principles of solid phase peptide synthesis. The innovation requires replacement of one or more of the amino acids in a peptide sequence by novel building units of the general Formula: 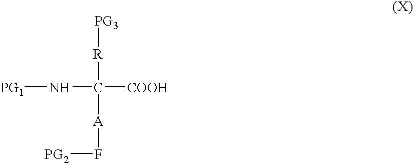
wherein the substitutents were defined hereinabove. A preferred embodiment of the present invention utilizes alkylene chains containing from two to ten carbon atoms.

The functional groups to be used for cyclization of the peptide analog include but are not limited to:
a. Amines, for reaction with electrophiles such as activated carboxyl groups, aldehydes and ketones (with or without subsequent reduction), and alkyl or substituted alkyl halides.
b. Alcohols, for reaction with electrophiles such as activated carboxyl groups.
c. Thiols, for the formation of disulfide bonds and reaction with electrophiles such as activated carboxyl groups, and alkyl or substituted alkyl halides.
d. 1,2 and 1,3 Diols, for the formation of acetals and ketals.
e. Alkynes or Substituted Alkynes, for reaction with nucleophiles such as amines, thiols or carbanions; free radicals; electrophiles such as aldehydes and ketones, and alkyl or substituted alkyl halides; or organometallic complexes.
f. Carboxylic Acids and Esters, for reaction with nucleophiles (with or without prior activation), such as amines, alcohols, and thiols.
g. Alkyl or Substituted Alkyl Halides or Esters, for reaction with nucleophiles such as amines, alcohols, thiols, and carbanions (from active methylene groups such as acetoacetates or malonates); and formation of free radicals for subsequent reaction with alkenes or substituted alkenes, and alkynes or substituted alkynes.
h. Alkyl or Aryl Aldehydes and Ketones for reaction with nucleophiles such as amines (with or without subsequent reduction), carbanions (from active methylene groups such as acetoacetates or malonates), diols (for the formation of acetals and ketals).
i. Alkenes or Substituted Alkenes, for reaction with nucleophiles such as amines, thiols, carbanions, free radicals, or organometallic complexes.
j. Active Methylene Groups, such as malonate esters, acetoacetate esters, and others for reaction with electrophiles such as aldehydes and ketones, alkyl or substituted alkyl halides.

It will be appreciated that during synthesis of the peptide these reactive end groups, as well as any reactive side chains, must be protected by suitable protecting groups.

Suitable protecting groups for amines are alkyloxy, substituted alkyloxy, and aryloxy carbonyls including, but not limited to, tert butyloxycarbonyl (Boc), Fluorenylmethyloxycarbonyl (Fmoc), Allyloxycarbonyl (Aloc) and Benzyloxycarbonyl (Z).

Carboxylic end groups for cyclizations may be protected as their alkyl or substituted alkyl esters or thio esters or aryl or substituted aryl esters or thio esters. Examples include but are not limited to tertiary butyl ester, allyl ester, benzyl ester, 2-(trimethylsilyl)ethyl ester and 9-methyl fluorenyl.

Thiol groups for cyclizations may be protected as their alkyl or substituted alkyl thio ethers or disulfides or aryl or substituted aryl thio ethers or disulfides. Examples of such groups include but are not limited to tertiary butyl, trityl(triphenylmethyl), benzyl, 2-(trimethylsilyl)ethyl, pixyl(9-phenylxanthen-9-yl), acetamidomethyl, carboxy-methyl, 2-thio-4-nitropyridyl.

It will further be appreciated by the artisan that the various reactive moieties will be protected by different protecting groups to allow their selective removal. Thus, a particular amino acid will be coupled to its neighbor in the peptide sequence when the N^{α} is protected by, for instance, protecting group A. If an amine is to be used as an end group for cyclization in the reaction scheme the N^{ω} will be protected by protecting group B, or an ε amino group of any lysine in the sequence will be protected by protecting group C, and so on.

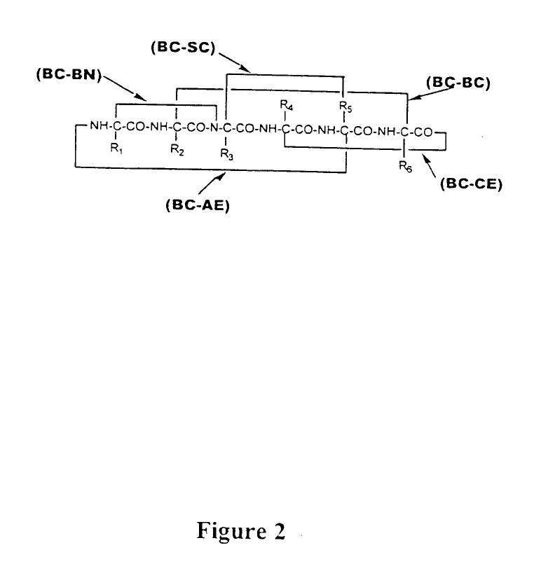

The coupling of the amino acids to one another is performed as a series of reactions as is known in the art of peptide synthesis. Novel building units of the invention, namely the C^{α}-ω functionalized amino acid derivatives are incorporated into the peptide sequence to replace one or more of the amino acids. As depicted in FIG. 2, modes of C-backbone cyclization are for example: BC-BN=backbone C to backbone N; BC-SC=back-bone C to side chain; BC-BC=backbone C to backbone C; BC-CE=backbone C to carboxy end; BC-AE=backbone C to amino end.

As stated above, the procedures utilized to construct peptide analogs of the present invention from novel building units generally rely on the known principles of peptide synthesis. However, it will be appreciated that accommodation of the procedures to the bulkier building units of the present invention may be required. Coupling of the amino acids in solid phase peptide chemistry can be achieved by means of a coupling agent such as but not limited to dicyclohexycarbodiimide (DCC), bis(2-oxo-3-oxazolidinyl) phosphinic chloride (BOP-Cl), benzotriazolyl-N-oxytrisdimethyl-aminophosphonium hexafluoro phosphate (BOP), 1-oxo-1-chlorophospholane (Cpt-Cl), hydroxybenzotriazole (HOBT), or mixtures thereof.

It has now been found that coupling of the bulky building units of the present invention may require the use of additional coupling reagents including, but not limited to: coupling reagents such as PyBOP® (Benzotriazole-1-yl-oxy-tris-pyrrolidino-phosphonium hexafluorophosphate), PyBrOP® (Bromo-tris-pyrrolidino-phosphonium hexafluorophosphate), HBTU (2-(1H-Benzotriazole-1-yl)-1,1,3,3-tetramethyluronium hexafluorophosphate), HBTU (2-(1H-Benzotriazole-1-yl)-1,1,3,3-tetramethyluronium tetrafluoroborate).

Novel coupling chemistries may be used, such as pre-formed urethane-protected N-carboxy anhydrides (UNCA's) and pre-formed acyl fluorides. Said coupling may take place at room temperature and also at elevated temperatures, in solvents such as toluene, DCM (dichloromethane), DMF (dimethylfomamide), DMA (dimethylacetamide), NMP (N-methylpyrrolidinone) or mixtures of the above.

The following examples are presented in order to more fully illustrate certain embodiments of the invention. They should in no way be construed, however, as limiting the broad scope of the invention.

### EXPERIMENTAL DETAILS SECTION

### Example 1

### Synthesis of C^{α}ω-Functionalized-alkyl-amino acid Building Blocks

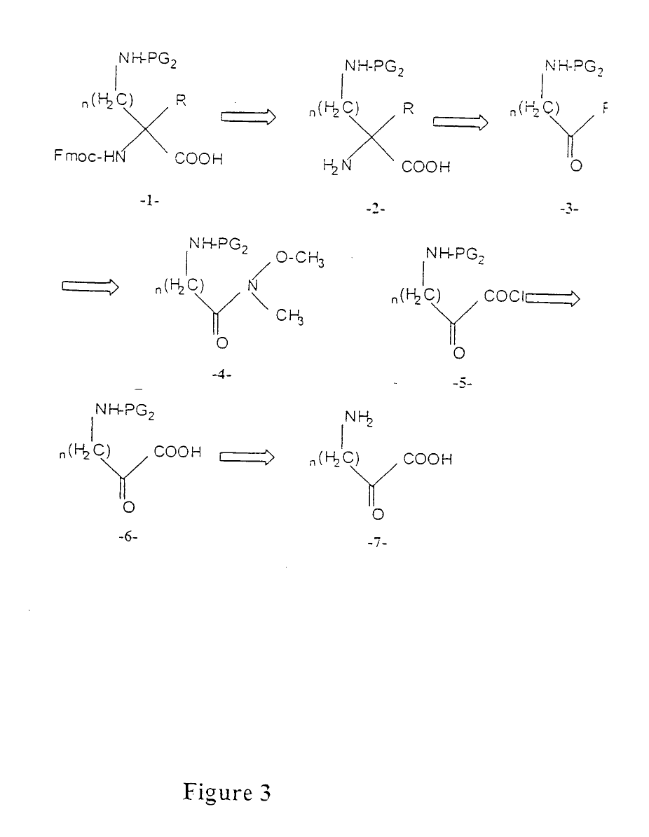

The synthesis of C^{α}ω-Functionalized-alkyl-amino acid building blocks was carried out in accordance with the retro synthetic scheme depicted in FIG. 3. The Strecker synthesis [Strecker, A., (1850) Liebigs Ann Chem, 75, 27] was chosen as a route of synthesis because of its compatibility with preparative scale synthesis and low price reagents. According to the retro synthetic scheme, asymmetric ketones 3 are needed as a substrate for the Strecker synthesis. These ketones were synthesized by the Weinreb reaction [Sibi, M. P.; Stressman, C. S.; Schultz, J.; Christensen, J. W.; Lu, J.; Marvin, M., (1995) Syn. Commun, 28, 1255] from the N-methoxy-N-methylamide derivative of carboxylic acids 4. The N-methoxy-N-methylamide derivatives were synthesized from the acyl chloride 5, that were prepared from the corresponding protected amino acid 6. The primary synthones were, therefore, a series of ω-amino acids 7 (n=1-6 are glycine, β-alanine, γ-amino butyric acid etc.).

For simpler monitoring of the progress of the synthesis by NMR spectroscopy, Applicants chose the benzyl group as the R group. Correspondingly, because of its comparatively low price, and simple structure that can be easily identified by NMR spectroscopy, Applicants used β-alanine (n=2) as a first synthone. The synthetic goal was not only to produce the C^{α}alkylated amino acid, but also to protect the two amine groups with two orthogonal protecting groups, that one of them, on the N^{α} should be the 9-fluorenylmethyloxycarbonyl group (Fmoc), so that the product will be suitable for solid phase synthesis (SPS) using the Fmoc chemistry. Thus, the ω-amine has to be protected prior to the protection of the α amine.

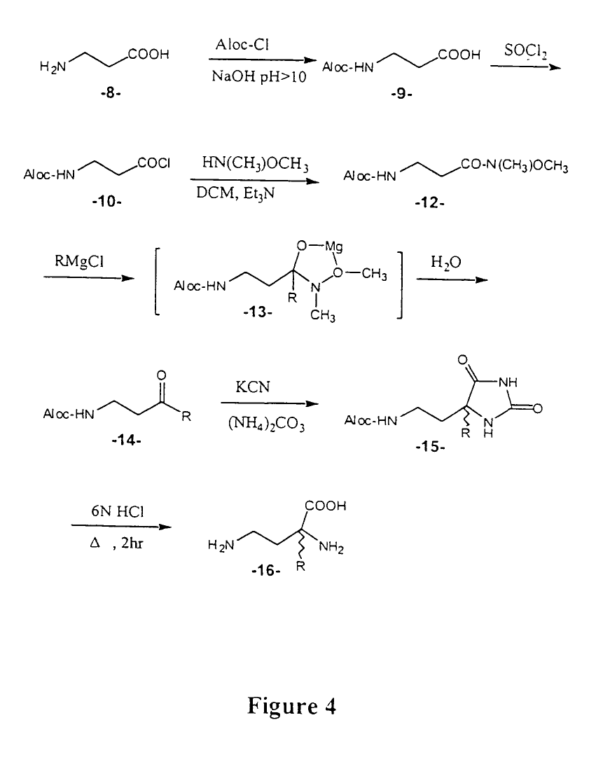

As shown in FIG. 4, the first step was the protection of the ω-amine of 7 with PG₂ which will be compatible with Fmoc SPS chemistry. The allyloxycarbonyl (Aloc) group [Stevens, C. M.; Watanabe, R., (1950) J. Am. Chem. Soc., 72, 725-727] as PG₂ was chosen because of the ease of its incorporation and deprotection and its stability to conditions employed in the next steps. The next step was to activate the carboxyl group for the coupling to the N-methoxy-N-methylamide. This was achieved by the conversion of the carboxylic acid function to the acyl chloride 10 using thionyl chloride. The chloride 10 was further coupled with N,O-dimethylhydroxylamine 11 in the presence of EtN₃. The yields of N-methoxy-N-methylamide 12 were low at the beginning (36-50%), and were then optimized to 76-90%. Coupling the carboxylic acid with N,O-dimethylhydroxylamine using PyBop as the coupling reagent [Fehrentz, J. A.; Castro, B., (1983) Synthesis, 676-678] gave low yields (25%). Another method, using Triphosgene (bis-trichlorocarbonate) as the coupling agent gave 58% yield.

The N-methoxy-N-methylamide 13 was then reacted with Grignard reagent to produce the asymmetric ketone 14 [Nahm, S; Weinreb, S. M., (1981) Tetrahedron Lett., 22, 3815-3818] in good yields. The ketone was treated with potassium cyanide and ammonium carbonate by the Bucherer-Bergs reaction to form the analogous hydantoin 15 [Stephani, R. A.; Rowe, W. B.; Gass, J. D.; Meister, A., (1972) Biochemistry, 11, 4094-4100]. The next step, hydrolysis of the hydantoin to the ω-Aloc protected amino acid 16 was troublesome. A series of attempts to hydrolyze the hydantoin in acidic or alkaline media at moderate temperatures [Ware, E., (1950) Chem. Rev., 403-470], yielded hydrolysis accompanied with Aloc deprotection or no hydrolysis at all. No selective reaction conditions were found for the hydrolysis. Hence, the hydantoin ring was hydrolyzed simultaneously with Aloc deprotection. Two hours reflux in 6 N HCl provided the amino acid 16.

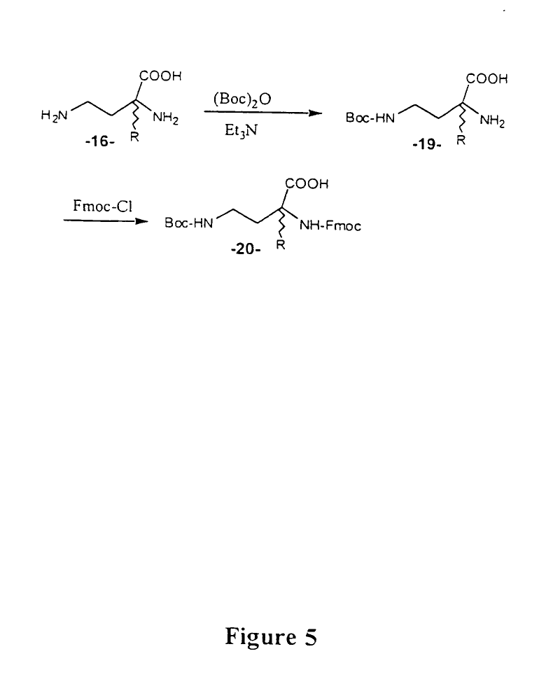

Next, the regioselective protection of the co)-amine was achieved by two strategies. The first involved mono Boc protection of the less steric hindered ω amine, with the bulky Boc group to produce 19. The product was confirmed by NMR. Fmoc protection of 19 gave the BU 20 in very low yield (FIG. 5).

In the second approach, N^{ω}-Aloc protection via complexation of the α-amino and the carboxylic acid groups with copper [Crivici, A.; Lajoie, G., (1993) Synth. Commun., 23, 49-53; Kurtz, A. C., (1938) J. Biol. Chem., 122, 477-484]. The complex we obtained did not dissolve in any solvent (water, DCM, alcohol, DMF etc.).

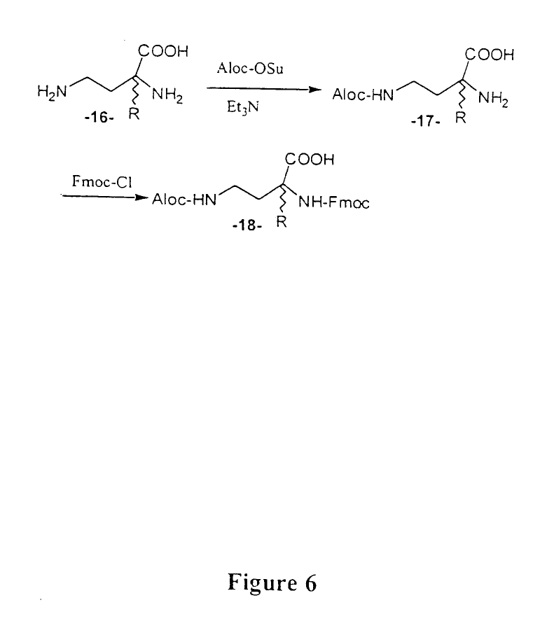

Thus, the ω-amine was protected with Aloc using a less reactive reagent then allylchloroformate. For this purpose allyloxycarbonyl succinimidyl carbonate (Aloc-OSu) was used [Blaakmeer, J.; Tijsse-Klasen, T.; Tesser, G. I., (1991) Int. J. Pept. Protein Res., 73, 556-564]. The Aloc-OSu was reacted with the 16 to furnish 17 in a good yield. The last step was to protect the N^{α} with Fmoc to produce 18 (FIG. 6).

The same procedures shown schematically in FIGS. 4 and 6 were used to prepare the following Fmoc-C^{α}(Aloc-amino alkyl)amino acids

| R | n = |
|---|---|
| Bzl | 2 |
| Iso-propyl | 3 |
| ethyl | 5 |

General Methods. All reactions of organometallic reagents were performed in flame-dried glassware under nitrogen. Solutions of these materials were transferred with hypodermic needles. Tetrahydrofuran (THF) was distilled from dark-blue or dark-purple solutions of sodium benzophenone radical anion or dianion under argon.

The 1H NMR spectra were recorded a Bruker 300 MHz machine. FAB-MS and ES-MS analysis was performed in the National center for MS, Technion. Haifa. Israel. Flash chromatography was performed using Merck silica gel.

Aloc-NH(CH₂)n-CO₂H. (1 mol) of H₂N—(CH₂)nCO₂H was dissolved in 250 ml. of 4N sodium hydroxide. The solution was cooled in an ice bath and treated with 138 ml. (1.3 mol) of allylchloroformate and 250 ml. of 4N sodium hydroxide added in eight portions with vigorous shaking for a few minutes after each addition. The pH was adjusted to 10 with 4 N sodium hydroxide solution (about 100 ml. were added). Reaction progress was detected by TLC (CHCl₃:MeOH 4:1). The reaction mixture was stirred for 24 hours before diluted with 450 ml. of water. The solution was washed three times with 300 ml. of petroleum ether. The aqueous layer was acidified to pH=1 with conc. hydrochloride acid and then was extracted three times with ethyl acetate. Ethyl acetate was dried over magnesium sulfate, and then was evaporated in vacuum to yield the desired product Aloc-NH—(CH₂)ₙ—CO₂H as a clear oil. No further purification was needed for the next step.

| | n = | | |
|---|---|---|---|
| | 2 | 3 | 5 |
| MW | 173.17 | 187.2 | 215.25 |
| | Clear oil | Clear oil | White solid |
| 1H NMR (CDCl₃, | 2.61(t, 2H), 3.74(q, 2H), | | |
| TMS) | 4.57(d, 2H), 5.26(dd, 2H), | | |
| | 5.93(m, 1H) | | |
| Yield (%) | 96 | 48.7 | 100 |

Aloc-NH—(CH₂)ₙ—CON(Me)OMe. A mixture of 0.1 mol of Aloc-NH—(CH₂)ₙ—CO₂H, 520 ml. of methylene chloride dried on calcium chloride, and 72.6 ml (1 mol, 10 eq) of thionyl chloride, was refluxed under argon atmosphere. After two hours 36.3 ml. Of thionyl chloride (5 eq) were added, and the solution was refluxed for additional one hour. After the solution was cooled to room temperature, the solvent and excess of thionyl chloride was removed by evaporation. 500 ml. of methylene chloride dried on calcium chloride was added and evaporated again. This addition and evaporation was repeated three more times, to get rid of all the excess of thionyl chloride. The yellow oil was dissolved in 500 ml of methylene chloride dried on calcium chloride. 10.73 gr. (0.11 mol) of N,O-dimethyl hydroxylamine hydrochloride were added to the solution, and the solution was stirred and cooled with an ice bath. Triethyl amine was added, enough to make the solution alkaline (about 30 ml.). The solution was stirred for one hour. Most of the solvent was evaporated, and the residue was partitioned between 200 ml. of brine and 200 ml. 1:1 mixture of ether and methylene chloride. The organic layer was dried with sodium sulfate, and evaporated to obtain the hydroxy amide. The residue can be used for the next step without further purification.

| | n = | | |
|---|---|---|---|
| | 2 | 3 | 5 |
| M.W. | 216.024 | 230.26 | 258.32 |
| 1H NMR (CDCl₃) | 2.65(t, 2H), | | |
| | 3.17(s, 3H) | | |
| | 3.46(q, 2H) | | |
| | 3.66(2, 3H) | | |
| | 4.54(d, 2H) | | |
| | 5.2(dd, 2H) | | |
| | 5.89(m, 1H) | | |
| Yield (%) | 86 | 39.6 | 68.5 |

Aloc-NH—(CH₂)ₙ—COR. To a solution of 21.6 gr. (0.1 mol) of Aloc-NH—(CH₂)ₙ—CON(Me)OMe in 500 ml. of dry THF in an ice bath were added 200 ml. of 2M RMgCl in THF (4 eq). The reaction mixture was stirred at 0° c. until TLC (petroleum ether:ethyl acetate 1:1) showed no starting amide. The reaction mixture was poured to a precooled 5% hydrochloric acid in ethanol (500 ml) (pH should be acidic). Most of the solvent was evaporated and the crude residue was partitioned between 250 ml. of brine and 250 ml. 1:1 mixture of ether and methylene chloride. The organic layer was dried over sodium sulfate and evaporated in vacuum. The product was purified by flash chromatography, affording the pure ketone.

| | n =, R = | | |
|---|---|---|---|
| | | n = 3, | |
| | n = 2, R = Bzl | R = Isobutyl | n = 5, R = Ethyl |
| M.W. | 247.29 | 230.26 | 258.32 |
| 1H NMR (CDCl₃) | 2.70(t, 2H), | | |
| | 3.38(q, 2H), | | |
| | 3.69(s, 2H), | | |
| | 4.52(d, 2H), | | |
| | 5.19(dq, 2H), | | |
| | 5.27(dq, 2H,) | | |
| | 5.88(m, 1H), | | |
| | 7.21(m, 5H). | | |
| yield | 70 | 33 | 31.8 |

5-Aloc(CH₂)ₙ-5-alkylhydantoin. 0.1 mol of Aloc-NH—(CH₂)ₙ—COR were dissolved in a 100 ml. 1:1 mixture of ethyl alcohol and water. 38.4 gr. (0.4 mol. 4 eq) of ammonium carbonate was added with stirring to control foaming. Then 17.32 gr. (0.266 mol, 2.6 eq) of potassium cyanide was added and the mixture was stirred and heated to 55-60° C. for 6 hours. The mixture was cooled to 0° c. for 30 minutes, and acidified with concentrated hydrochloric acid (ca. 100 ml), and was left in the hood overnight. The reaction mixture was extracted three times with 50 ml. of ethyl acetate. The organic residue was dried over magnesium sulfate and evaporated in vacuum to obtain an orange oil. after a while, white crystals were formed. The crystals were washed with chloroform or ether, to obtain the hydantoin.

| | n =, R = | | |
|---|---|---|---|
| | | n = 3, | |
| | n = 2, R = Bzl | R = Isobutyl | n = 5, R = Ethyl |
| M.W. | 317.35 | | 297.36 |
| 1H NMR (CDCl₃) | 1.9(dm, 2H), | | |
| | 2.85(dd, 2H), | | |
| | 3.00(dm, 2H), | | |
| | 4.46(d, 2H), | | |
| | 5.21(dd, 2H) | | |
| | 5.89(m, 1H). | | |
| yield | 47.6 | | 79.5 |

Hydrolysis of the hydantoin. 5-Aloc(CH₂)ₙ-5-alkylhydantoin (0.03308 mol) were added to 45 ml of 6N HCl solution, and refluxed for 2.5 hours. During this time, the solution became yellow, and all the solid was dissolved. The solution was cooled to 0° C., until a white solid was separated. The solid was collected by filtration, and dried in vacuum.

| | n =, R = | | |
|---|---|---|---|
| | | n = 3, | |
| | n = 2, R = Bzl | R = Isobutyl | n = 5, R = Ethyl |
| M.W. | 281.2 | | 261.29 |
| 1H NMR (CDCl₃) | 2.03(t, 2H), | | |
| | 2.80(dm, 2H), | | |
| | 2.88(q, 2H), | | |
| | 7.15(m, 5H), | | |
| | 8.14(s, 3H), | | |
| | 8.29(s, 1H), | | |
| | 10.42(s, 1H). | | |
| yield | 87 | | 75.5 |

Selective protection with Aloc-OSu. The unprotected building unit (0.0259 mol) was dissolved in 80 ml TDW. 3.6 ml of triethylamine was added. To the stirred solution was slowly added a solution of 4.6 gr. Aloc-Osu dissolved in 115 ml of acetonitrile (about 1 hour). The reaction mixture was monitored by TLC until no more Aloc-OSu was detected (two days). The reaction mixture was washed 3 times with 100 ml petroleum ether. The aqueous layer was then evaporated to yield an oily residue. The oil was extracted to ethyl acetate, the organic solution was dried over MgSO₄, and evaporated resulting solid powder.

| | n =, R = | | |
|---|---|---|---|
| | | n = 3, | |
| | n = 2, R = Bzl | R = Isobutyl | n = 5, R = Ethyl |
| M.W. | 292.3 | | 272.44 |
| 1H NMR (CDCl₃) | 1.87(dm, 2H), | | |
| | 2.85(dd, 2H), | | |
| | 3.02(dm, 2H), | | |
| | 4.46(d, 2H), | | |
| | 5.21(dd, 2H) | | |
| | 5.90(m, 1H). | | |
| yield | 41.73 | | 97.5 |

Protection of the N^{α} with Fmoc. 0.0309 mol of 2 was dissolved in 200 acetonitrile. 11.5 ml of diisopropylethylamine (DIEA) was added, and the solution was cooled to 0° c. A solution of 8 gr. Fmoc-Cl in 70 ml of DCM was added to the stirred reaction. The solution was stirred for 3 hours before 70 ml of DCM was added. The organic layer was washed 3 times with 1N HCl solution, and then 3 times with saturated NaCl. The organic layer was dried over Na₂SO₄, filtered and evaporated. The resulting yellowish solid was purified over silica. The collected fractions were evaporated, and the N^{α}-Fmoc, N^{α}-Aloc protected building unit was obtained as a white solid.

| | n =, R = | | |
|---|---|---|---|
| | | n = 3, | n = 5, |
| | n = 2, R = Bzl | R = Isobutyl | R = Ethyl |
| M.W. | 514.56 | | 494.67 |
| 1H NMR (CDCl₃) | 2.18(dm, 2H), | | |
| | 3.10(dd, 2H), | | |
| | 3.34(m, 2H), | | |
| | 4.26(t, 1H), | | |
| | 4.45(m, 2H), | | |
| | 4.55(d, 2H), | | |
| | 5.22(dd, 2H), | | |
| | 5.87(m, 1H), | | |
| | 7.18-7.44(m, 9H), | | |
| | 7.68(d, 2H), | | |
| | 7.76(d, 2H). | | |
| yield | 29 | | 81 |

It will be appreciated by a person skilled in the art that the present invention is not limited by what has been particularly shown and described hereinabove. Rather, the scope of the invention is defined by the claims which follow.

1. A cyclized peptide analog comprising, a sequence of amino acids that incorporates at least one building unit, wherein said building unit is a modified amino acid having an alpha-carbon atom of the peptide backbone attached through an optional spacer to a functional group selected from amine, thio, oxy, and carboxy, wherein said building unit is joined to another amino acid within said sequence to form a bridging group comprising a disulfide, amide, thioether, thioester, imine, ether, ester or an alkene.

2. The cyclized peptide analog cyclized peptide analog of claim 1, comprising two building units joined together to form a cyclic structure.

3. The cyclized peptide analog of claim 1, comprising one building unit.

4. The cyclized peptide analog of claim 1, wherein said building unit is joined to an amino acid is located at the carboxy end of the peptide sequence.

5. The cyclized peptide analog of claim 1, wherein said building unit is joined to an amino acid is located at the amino end of the peptide sequence.

6. The cyclized peptide analog of claim 1, wherein said building unit is joined to an amino acid through the side chain of said amino acid.

7. The cyclized peptide analog of claim 1, wherein said building unit is joined to an amino acid through the backbone nitrogen atom of said amino acid.

8. The cyclized peptide analog of claim 1, represented by the structure of Formula (I): 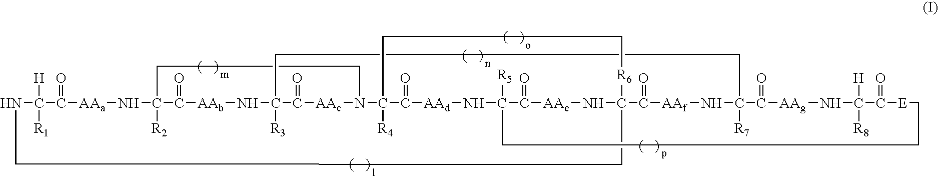
wherein
a, b, c, d, e, f and g are independently of each other an integer from 1 to 8 or zero;
l, m, n, o and p are independently of each other zero or 1, wherein at least one of l, m, n, o or p is 1;
each AA designates an amino acid residue wherein the amino acid residues may be the same or different;
E designates an oxygen, an amino, a carboxyl protecting group, wherein E is optionally bound to a solid support, or CO-E can be reduced to CH₂O;
R₁-R₈ are independently of each other hydrogen or an amino acid side-chain optionally bound with a protecting group; and
the lines designate a bridging group of the Formula:
(i) —X-M-Y—W-Z- or (ii) —X-M-Z-
wherein
M and W are independently of each other a disulfide, amide, thioether, thioester, imine, ether, ester or an alkene; and
X, Y and Z independently of each other an unsubstituted or substituted alkylene, alkenylene, alkynylene, arylene, cycloalkylene, alkylarylene, heterocycloalkylene or heteroarylene.

9. The cyclized peptide analog of claim 8, wherein l is one.

10. The cyclized peptide analog of claim 8, wherein m is one.

11. The cyclized peptide analog of claim 8, wherein n is one.

12. The cyclized peptide analog of claim 8, wherein o is one.

13. The cyclized peptide analog of claim 8, wherein p is one.

14. The cyclized peptide analog of claim 8, wherein the group CO-E is CH₂O.

15. The cyclized peptide analog of claim 8, wherein R₁-R₈ are independently of each other CH₃—, (CH₃)₂—CH—, (CH₃)₂—CHCH₂—, CH₃CH₂CH(CH₃)—, CH₃S(CH₂)₂—, HOCH₂—, CH₃CH(OH)—, HSCH₂—, NH₂C(═O)CH₂—, NH₂C(═O), (CH₂)₂—, NH₂(CH₂)₃—, HOC(═O)CH₂—, HOC(═O)(CH₂)₂—, NH₂(CH₂)₄—, C(NH₂)₂NH(CH₂)₃—, HO-phenyl-CH₂—, benzyl, methylindole, or methylimidazole.

16. The cyclized peptide analog of claim 8, represented by the structure of Formula (II):

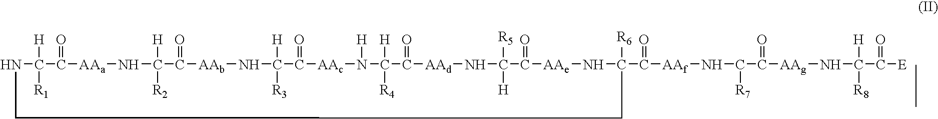

17. The cyclized peptide analog of claim 8, represented by the structure of Formula (III):

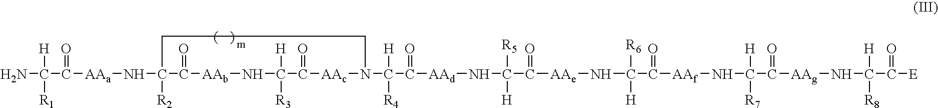

18. The cyclized peptide analog of claim 8, represented by the structure of Formula (IV):

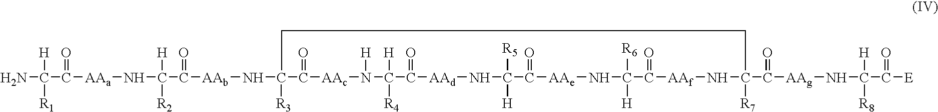

19. The cyclized peptide analog of claim 8, represented by the structure of Formula (V):

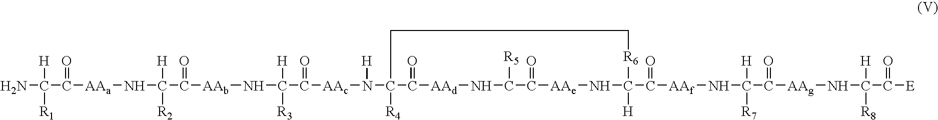

20. The cyclized peptide analog of claim 8, represented by the structure of Formula (VI):

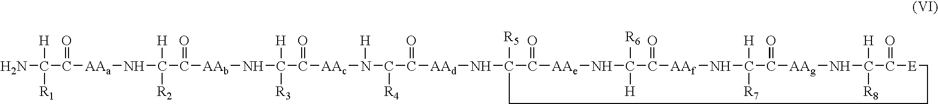

21. A pharmaceutical composition comprising a cyclized peptide analog according to claim 1 and a pharmaceutically acceptable carrier or diluent.

22. A pharmaceutical composition comprising a cyclized peptide analog according to claim 8 and a pharmaceutically acceptable carrier or diluent.

23. A pharmaceutical composition comprising a cyclized peptide analogs according to claim 16 and a pharmaceutically acceptable carrier or diluent.

24. A pharmaceutical composition comprising a cyclized peptide analog according to claim 17 and a pharmaceutically acceptable carrier or diluent.

25. A pharmaceutical composition comprising a cyclized peptide analog according to claim 18 and a pharmaceutically acceptable carrier or diluent.

26. A pharmaceutical composition comprising backbone cyclized peptide analog according to claim 19 and a pharmaceutically acceptable carrier or diluent.

27. A pharmaceutical composition comprising backbone cyclized peptide analog according to claim 20 and a pharmaceutically acceptable carrier or diluent.

28. A method of making an ω-functionalized amino acid derivative of the general Formula X: 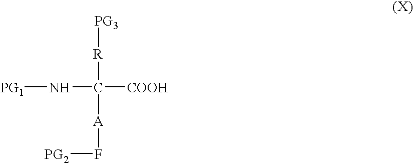
wherein
A is a spacer group selected from unsubstituted or substituted alkylene, alkenylene, alkynylene, arylene, cycloalkylene, alkylarylene, heterocycloalkylene or heteroarylene;
F is a functional group selected from amine, thio, oxy, or carboxy;
PG₁, PG₂ and PG₃ are independently of each other hydrogen or a protecting group selected from alkyloxy, substituted alkyloxy, or aryloxy carbonyls; and
R is a side chain of an amino acid;
said method comprising the steps of:
reacting a carboxylic acid derivative of formula VII with a reagent containing a nucleophillic R group, to produce compound VIII;
converting compound VIII to amino acid derivative IX; and
optionally protecting the amino group of compound IX;
thereby preparing said (s-functionalized amino acid derivative X.

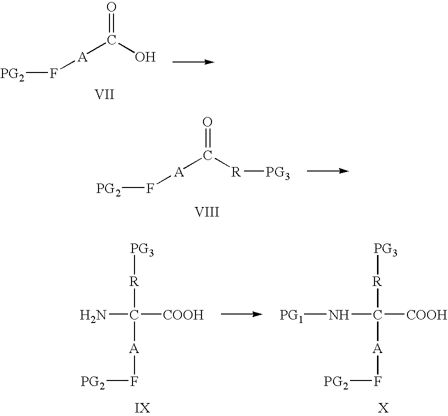

29. The method of claim 28, wherein PG₁ is an amino protecting group selected from Ada, Aloc, Allyl, Boc, Bzl, Fmoc, OBzl, OEt, OMe, Tos, Trt and benzyloxycarbonyl

30. The method of claim 28, wherein PG₂ is a functional group protecting group selected from Ada, Aloc, Allyl, Boc, Bzl, Fmoc, OBzl, OEt, OMe, Tos, Trt and benzyloxycarbonyl

31. The method of claim 28, wherein PG₃ is a side chain protecting group selected from Ada, Aloc, Allyl, Boc, Bzl, Fmoc, OBzl, OEt, OMe, Tos, Trt and benzyloxycarbonyl.

32. The method of claim 28, wherein said compound containing a nucleophillic R group is represented by the structure RM(L)ₓ wherein M is a metal, L is a leaving group and X is zero or 1.

33. The method of claim 28, wherein the step of converting carboxylic acid VU to compound VIII comprises the steps of
converting said carboxylic acid into a reactive derivative thereof; and
reacting said reactive carboxylic acid derivative with a compound containing a nucleophillic R group.

34. The method according to claim 28, wherein the step of converting carboxylic acid VII to compound VII is carried out under conditions of the Weinreb reaction.

35. The method of claim 28, wherein the step of converting compound VIII to compound IX is carried out under conditions of the Strecker synthesis.

36. A method for the preparation of a cyclized peptide analog of the general Formula (I): 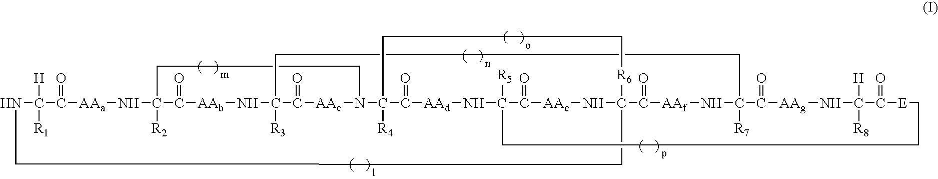
wherein
a, b, c, d, e and f are independently of each other an integer from 1 to 8 or zero;
l, m, n, o and p are independently of each other zero or 1, wherein at least one of l, m, n, o or p is 1;
each AA designates an amino acid residue wherein the amino acid residues may be the same or different;
E designates an oxygen, an amino, a carboxyl protecting group, wherein E is optionally bound to a solid support, or CO-E can be reduced to CH₂O;
R₁-R₈ are independently of each other hydrogen or an amino acid side-chain optionally bound with a protecting group; and
the lines designate a bridging group of the Formula:
(i) —X-M-Y—W-Z- or (ii) —X-M-Z-
wherein
M and W are independently of each other a disulfide, amide, thioether, thioester, imine, ether, ester or an alkene; and
X, Y and Z independently of each other an unsubstituted or substituted alkylene, alkenylene, alkynylene, arylene, cycloalkylene, alkylarylene, heterocycloalkylene or heteroarylene,
said method comprising the step of incorporating at least one C^{α}-ω-functionalized derivatives of amino acids of Formula (X) into a peptide sequence and subsequently selectively cyclizing the functional group with one of the amino acids in said peptide. 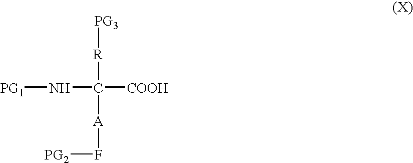
wherein
A is a spacer group selected from unsubstituted or substituted alkylene, alkenylene, alkynylene, arylene, cycloalkylene, alkylarylene, heterocycloalkylene or heteroarylene;
F is a functional group selected from amine, thio, oxy, or carboxy;
PG₁, PG₂ and PG₃ are independently of each other hydrogen or a protecting group selected from alkyloxy, substituted alkyloxy, or aryloxy carbonyls; and
R is a side chain of an amino acid.

37. The method of claim 36, wherein said C^{α}-ω-functionalized amino acid is cyclized with an amino acid located at the carboxy end of the peptide sequence.

38. The method of claim 36, wherein said C^{α}-ω-functionalized amino acid is cyclized with an amino acid located at the amino end of the peptide sequence.

39. The method of claim 36, wherein two of said C^{α}-ω-functionalized amino acids are cyclized to form a cyclic structure.

40. The method of claim 36, wherein said C^{α}-ω-functionalized amino acid is cyclized with an amino acid through the backbone nitrogen of said amino acid.

41. The method of claim 36, wherein said C^{α}-ω-functionalized amino acid is cyclized with an amino acid through the side chain of said amino acid.

42. The method of claim 36, wherein l is one.

43. The method of claim 36, wherein m is one.

44. The method of claim 36, wherein n is one.

45. The method of claim 36, wherein o is one.

46. The method of claim 36, wherein p is one.

47. The method of claim 36, wherein the group CO-E is CH₂O.

48. The method of claim 36, wherein E is bound to a solid support.

49. The method of claim 36, wherein R₁-R₈ are independently of each other CH₃—, (CH₃)₂——CH—, (CH₃)₂—CHCH₂—, CH₃CH₂CH(CH₃)—, CH₃S(CH₂)₂—, HOCH₂—, CH₃CH(OH)—, HSCH₂—, NH₂C(═O)CH₂—, NH₂C(═O), (CH₂)₂—, NH₂(CH₂)₃—, HOC(═O)CH₂—, HOC(═O)(CH₂)₂—, NH₂(CH₂)₄—, C(NH₂)₂NH(CH₂)₃—, HO-phenyl-CH₂—, benzyl, methylindole, or methylimidazole.

50. The method of claim 36, wherein said cyclized peptide analog is represented by the structure of Formula (II):

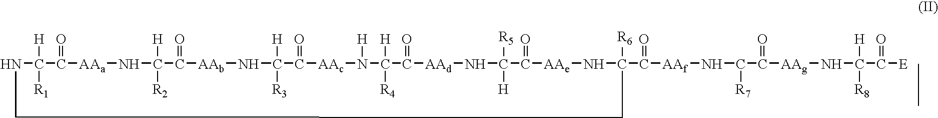

51. The method of claim 36, wherein said cyclized peptide analog is represented by the structure of Formula (III):

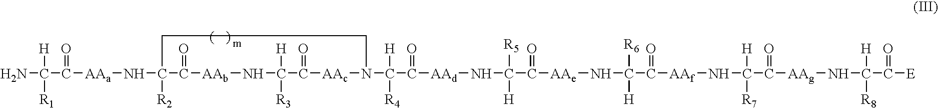

52. The method of claim 36, wherein said cyclized peptide analog is represented by the structure of Formula (IV):

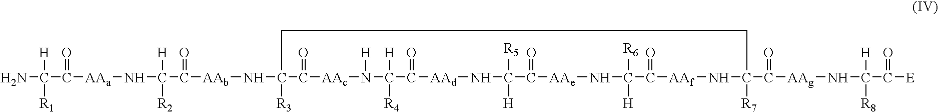

53. The method of claim 36, wherein said cyclized peptide analog is represented by the structure of Formula (V):

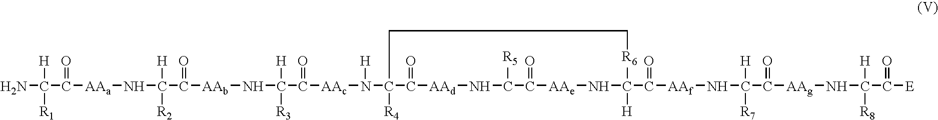

54. The method of claim 36, wherein said cyclized peptide analog is represented by the structure of Formula (VI): [chemical structure]
